(19) United States
(12) Patent Application Publication
(10) Pub. No.: US 2021/0121329 A1
(43) Pub. Date: Apr. 29, 2021

(54) HEATED WELDING LENS

(71) Applicant: Larry Poss, Elba, NE (US)

(72) Inventor: Larry Poss, Elba, NE (US)

(21) Appl. No.: 16/667,899

(22) Filed: Oct. 29, 2019

(51) Int. Cl. A61F 9/06; F16P 1/06

(52) U.S. Cl. CPC A61F 9/06; F16P 1/06

(57) ABSTRACT

An illustrated view of an exemplary heated welding lens device for eliminating fogging of a welding helmet is presented. The heated welding lens device is useful adjusting a temperature of a lens of the welding helmet such that the fogging that is common for temperature differentiation during welding. The heated welding lens device is useful for ensuring the safety, efficiency and comfort of a welder during a work shift or a welding activity.

### FIELD OF THE INVENTION

This invention relates to welding. More particularly, it relates to a mask for use while welding.

### BACKGROUND

Welding is a fabrication or sculptural process that joins materials, usually metals or thermoplastics, by using high heat to melt the parts together and allowing them to cool causing fusion. Welding is distinct from lower temperature metal-joining techniques such as brazing and soldering, which do not melt the base metal.

In addition to melting the base metal, a filler material is typically added to the joint to form a pool of molten material (the weld pool) that cook to form a joint that, based on weld configuration (butt, full penetration, fillet, etc.), can be stronger than the base material (parent metal). Pressure may also be used in conjunction with heat, or by itself, to produce a weld. Welding also requires a form of shield to protect the filler metals or melted metals from being contaminated or oxidized.

Many different energy sources can be used for welding, including a gas flame (chemical), an electric arc (electrical), a laser, an electron beam, friction, and ultrasound. While often an industrial process, welding may be performed in many different environments, including in open air, under water, and in outer space. Welding is a hazardous undertaking and precautions are required to avoid burns, electric shock, vision damage, inhalation of poisonous gases and fumes, and exposure to intense ultraviolet radiation.

Until the end of the 19th century, the only welding process was forged welding, which blacksmiths had used for millennia to join iron and steel by heating and hammering. Arc welding and oxy-fuel welding were among the first processes to develop late in the century, and electric resistance welding followed soon after. Welding technology advanced quickly during the early 20th century as the world wars drove the demand for reliable and inexpensive joining methods.

Following the wars, several modern welding techniques were developed, including manual methods like shielded metal arc welding, now one of the most popular welding methods, as well as semi-automatic and automatic processes such as gas metal arc welding, submerged arc welding, flux-cored arc welding and electroslag welding. Developments continued with the invention of laser beam welding, electron beam welding, magnetic pulse welding, and friction stir welding in the latter half of the century. Today, the science continues to advance. Robot welding is commonplace in industrial settings, and researchers continue to develop new welding methods and gain greater understanding of weld quality.

Welding can be dangerous and unhealthy if the proper precautions are not taken. However, using new technology and proper protection greatly reduces risks of injury and death associated with welding. Since many common welding procedures involve an open electric arc or flame, the risk of burns and fire is significant; this is why it is classified as a hot work process. To prevent injury, welders wear personal protective equipment in the form of heavy leather gloves and protective long-sleeve jackets to avoid exposure to extreme heat and flames. Synthetic clothing such as non-fireproofed polyester should not be worn since it will ignite and burn rapidly. Additionally, the brightness of the weld area leads to a condition called arc eye or flash burns in which ultraviolet light causes inflammation of the cornea and can burn the retinas of the eyes. Goggles and welding helmets with dark UV-filtering face plates are worn to prevent this exposure. Since the 2000s, some helmets have included a face plate which instantly darkens upon exposure to the intense UV light. To protect bystanders, the welding area is often surrounded with translucent welding curtains. These curtains, made of a polyvinyl chloride plastic film, shield people outside the welding area from the UV light of the electric arc, but cannot replace the filter glass used in helmets.

A welding helmet is a type of headgear used when performing certain types of welding to protect the eyes, face and neck from flash burn, ultraviolet light, sparks, infrared light, and heat.

Welding helmets are most commonly used with arc welding processes such as shielded metal arc welding, gas tungsten arc welding, and gas metal arc welding. They are necessary to prevent arc eye, a painful condition where the cornea is inflamed. Welding helmets can also prevent retina burns, which can lead to a loss of vision. Both conditions are caused by unprotected exposure to the highly concentrated ultraviolet and infrared rays emitted by the welding arc. Ultraviolet emissions from the welding arc can also damage uncovered skin, causing a sunburn-like condition in a relatively short period of welding. In addition to the radiation, gasses or splashes can also be a hazard to the skin and the eyes.

Most welding helmets include a window covered with a filter called a lens shade, through which the welder can see to work. In most helmets, the window may be made of tinted glass, tinted plastic, or a variable-density filter made from a pair of polarized lenses.

While the current welding helmets makes work simpler, quick and safe, it also means that the welding helmet spends more time on a welder's face during a work shift. As these helmets are large, heavy and thick, they also do a great job in retain heat. This makes the lends system prone to fogging due to the temperature differential across the helmet. In cold environments, the sudden heat on an exterior of the helmet versus the cold on an interior of the helmet can cause fogging as well. Such fogging not only makes seeing the weld pattern difficult, it also presents a safety hazard.

Accordingly, and in light of the foregoing, three is a need for a device that can eliminate the fogging of lenses in arc welding helmets.

### BRIEF DESCRIPTION OF THE DRAWINGS

FIG. 1 is an illustrated view of an exemplary heated welding lens device.

FIG. 2 is an illustrated view of a welding helmet with the heated welding lens device shown in FIG. 1.

### DETAILED DESCRIPTION

The phrases “in one embodiment,” “in various embodiments,” “in some embodiments,” and the like are used repeatedly. Such phrases do not necessarily refer to the same embodiment. The terms “comprising,” “having,” and “including” are synonymous, unless the context dictates otherwise. Such terms do not generally signify a closed list.

“Above,” “adhesive,” “affixing,” “any,” “around,” “both,” “bottom,” “by,” “comprising,” “consistent,” “customized,” “enclosing,” “friction,” “in,” “labeled,” “lower,” “magnetic,” “marked,” “new,” “nominal,” “not,” “of,” “other,” “outside,” “outwardly,” “particular,” “permanently,” “preventing,” “raised,” “respectively,” “reversibly,” “round,” “square,” “substantial,” “supporting,” “surrounded,” “surrounding,” “threaded,” “to,” “top,” “using,” “wherein,” “with,” or other such descriptors herein are used in their normal yes-or-no sense, not as terms of degree, unless context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to, or combined, without limiting the scope to the embodiments disclosed herein.

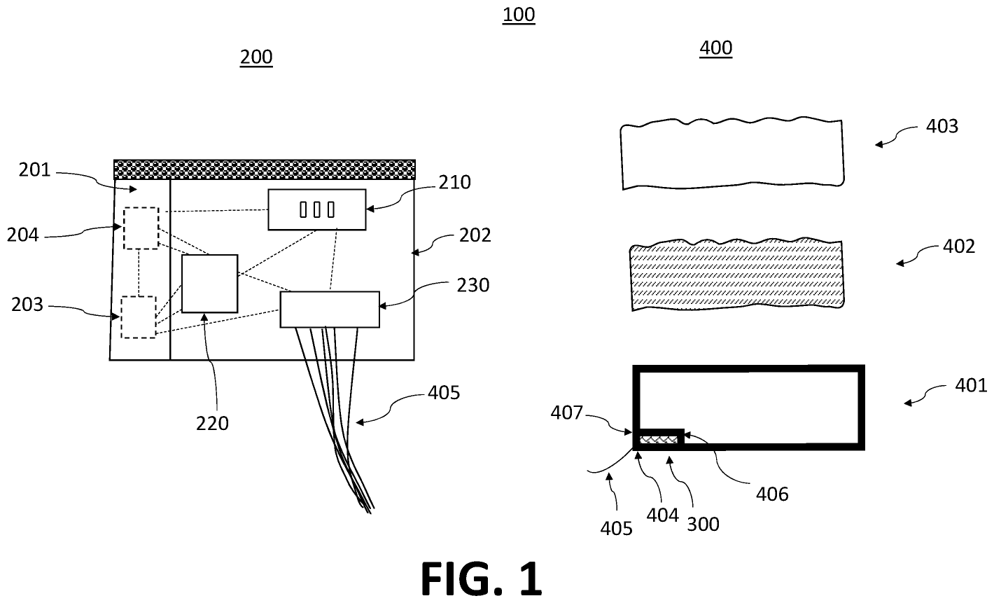

Referring to FIG. 1, an illustrated view of an exemplary heated welding lens device 100 for eliminating fogging of a welding helmet is presented. The heated welding lens device 100 is useful adjusting a temperature of a lens of the welding helmet such that the fogging that is common for temperature differentiation during welding. The heated welding lens device 100 is useful for ensuring the safety, efficiency and comfort of a welder during a work shift or a welding activity.

The heated welding lens device 100 has a control pack 200 and a heating element 300. The control pack 200 is preferably made of fire resistant and fire-retardant materials.

A lens assembly 400 has a front lens 403, a dark welding filter 402 and a third lens 401. The heating panel 300 is coupled to a first corner 404 of a third lens 401. The third lens 401 is coupled to a dark welding filter 402 and the dark welding filter 402 is further coupled to a front lens 403. The third lens is preferably clear 401. The heating panel 300 is electrically and communicatively coupled to the control pack 200 by one or more wires 405. A grommet 406 is coupled to an outside 407 of the heating panel 300.

The control pack 200 has an inside 201 and a front 202. The inside 201 of the control pack 200 has a power source 203 and a computing device 204. The power source 203 is useful for providing electrical current to the computing device 204, all components of the front 202 of the control pack 200, and to the heating panel 300.

The power source 203 is preferably a lithium polymer battery, however other types of power sources are hereby contemplated, including, but not limited to, disposable battery, rechargeable battery, ac/dc, etc. The power source 203 is preferably rechargeable. The power source 203 is coupled to a charging unit (not shown) by a charging cable (not shown). The charging unit is preferably a cigarette lighter of a car, however other charging units are hereby contemplated, including, but not limited to one and one-half (1.5) amp AC power supply converter, etc.

The front 202 of the control pack 200 has a temperature control panel 210, a power control 220 and a wiring port 230.

The temperature control panel 210 is useful for providing a temperature for the heating panel 300 of the third lens 401. The temperature control panel 210 is preferably composed of light-emitting diode (LED) lamps, however other types of lamps are hereby contemplated, including, but not limited to, liquid crystal display (LCD), pen light, etc. The temperature control panel 210 is preferably a touch screen thereby allowing for the temperature for the heating panel 300 to be set to, however the temperature control panel 210 may be any other type of screens. The temperature control panel 210 is electrically coupled to the power source 203 and is communicatively coupled to the computing device 204. The temperature control panel 210 is further coupled to the wiring port 230.

The power control 220 is useful for controlling the usage of the heated welding lens 100. The power control 220 is electrically coupled to the power source 203. Further, the power control 220 is electrically coupled to the wiring port 203. The power control 220 is preferably a switch for on/off settings, however other types of controls are hereby contemplated, including, but limited to, a button, a toggle switch, a touch screen, etc.

The one or more wires 405 are coupled to the wiring port 230. The one or more wires 405 are further coupled electrically and communicatively to the heating panel 300.

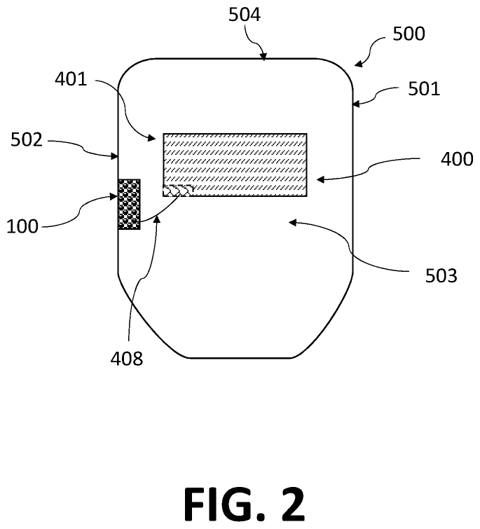

Moving now to FIG. 2, an illustrated view of a welding helmet 500 with the heated welding lens device shown in FIG. 1.

The helmet 500 has a mask 501 and a lens 400. The lens assembly 400 has been described in FIG. 1. The control pack 200 is coupled to an outer edge 502 of the helmet 500. The third lens 401 of the lens assembly 400 is coupled to the helmet 500 preferably between the middle portion 503 and the upper edge 504 of the helmet 500. The heating panel 300 is coupled to the wiring port 430 of the front 202 of the control pack 200 by a temperature control wire 408 of the one or more wires 405.

In the numbered clauses below, specific combinations of aspects and embodiments are articulated in a shorthand form such that (1) according to respective embodiments, for each instance in which a “component” or other such identifiers appear to be introduced (with “a” or “an,” e.g.) more than once in a given chain of clauses, such designations may either identify the same entity or distinct entities; and (2) what might be called “dependent” clauses below may or may not incorporate, in respective embodiments, the features of “independent” clauses to which they refer or other features described above.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A heated welding lens device for eliminating fogging of a lens of a welder's helmet, the device comprising:
a heating panel, the heating panel being coupled to a third lens of the helmet, wherein the heating panel having a grommet;
a control pack, the control pack having an inside and a front;
a power source, the power source being in the inside of the control pack;
a computing device, the computing device being in the inside of the control pack, wherein the computing device being electrically coupled to the power source;
a power control, the power control being coupled to the front of the control pack, and wherein the power control being coupled to the power source;
a temperature control panel, the temperature control panel being coupled to the front of the control pack, wherein the temperature control panel being electrically coupled to the power source, and wherein the temperature control panel being communicatively coupled to the computing device; and
a wiring port, the wiring port being coupled to the computing device, the power source, the power control and the temperature control panel, and wherein the wiring port having a temperature control wire, wherein the temperature control panel being communicatively coupled to the heat panel coupled to the third lens of the helmet.

2. The device of claim 1, wherein control box being made of a fire-resistant material.

3. The device of claim 1, wherein control box being made of a fire-retardant material.

4. The device of claim 1, wherein the third lens being clear.

5. The device of claim 1, wherein the power source being a lithium polymer battery.

6. The device of claim 1, wherein the power source being rechargeable.

7. The device of claim 1, wherein the power source further comprising a charging cable, the charging cable for coupling to a charging unit.

8. The device of claim 7, wherein the charging unit being a one and one-half (1.5) amp AC converter.

9. The device of claim 7, wherein the charging unit being a cigarette lighter of a car.

10. The device of claim 1, wherein the temperature control panel having light emitting diode (LED) lamps.

11. The device of claim 1, wherein the temperature control panel being a touch screen.

12. The device of claim 1, wherein the power control being an on/off switch.
